(19) United States
(12) Patent Application Publication
(10) Pub. No.: US 2026/0014187 A1
(43) Pub. Date: Jan. 15, 2026

(54) Stable Composition For Storing And Transporting Single Strand Nucleic Acid Material

(71) Applicant: Life Magnetics, Inc., Ann Arbor, MI (US)

(72) Inventors: Kevin Hagedorn, Northville, MI (US); Rishabh Kala, Portage, MI (US)

(21) Appl. No.: 18/994,484

(22) PCT Filed: Jul. 17, 2023
(86) PCT No.: PCT/US2023/070334

§ 371 Date: Jan. 14, 2025

Related U.S. Application Data
(60) Provisional application No. 63/389,590, filed on Jul. 15, 2022.
(60) Provisional application No. 63/413,533, filed on Oct. 5, 2022.

(51) Int. Cl. A61K 31/7105; A61K 9/51; C12Q 1/6806

(52) U.S. Cl. CPC A61K 31/7105; A61K 9/5115; C12Q 1/6806

(57) ABSTRACT

A composition includes a plurality of biocompatible metal nanospheres. the biocompatible nanospheres each having an outer surface with elemental carbon connected thereto. The composition also includes a plurality of single nucleic acids strands, wherein at least a portion of the respective individual single nucleic acid stands are in coordinated connection with the nanospheres, and a carrier medium.

### CROSS-REFERENCES TO RELATED APPLICATIONS

This application claims priority to U.S. Provisional Patent Application Nos. 63/389,590, filed Jul. 15, 2022, and 63/413,533, filed Oct. 5, 2022, each of which is incorporated herein in its entirety by reference.

### TECHNICAL FIELD

Disclosed are compositions suitable for storing and/or transporting and/or delivering single strand nucleic acid material such as ribonucleic acid (RNA) material. Also disclosed are methods of producing such compositions and the storage/transport/delivery of RNA material.

### BACKGROUND

Various types of single-strand nucleic acids play important roles in research as well as in medical treatment, disease prevention and mediation. One such application is the use of messenger RNA (mRNA) for use as a vaccine or booster for resistance to diseases such a s SARS-CoV-2 in a recipient.

One challenge to the use of nucleic acid such as RNA, which is a type of single strand nucleic acid, in various biological and biomedical applications is the instability and/or reactivity of the nucleic acid strand. Long term stable storage of this material has been a continuing challenge. To date, storage, transport, and delivery of RNA material has been hampered by the extreme measures that need to be taken to ensure that at least a portion of the stored material remains viable during the storage interval as well as during transport and delivery.

This challenge has been evidenced in end-use applications including, but not limited to, mRNA vaccine delivery. In the challenges that are presented in obtaining viable active single strand nucleic acid material, vaccines formulated with single strand nucleic acid material must be kept stable for days or even weeks before being administered. Ribonucleic acids such as the mRNA employed in such materials break down easily and quickly unless kept as low temperatures. Given this, therapeutics using ribonucleic acid such a mRNA as well as their precursors must be transported and stored at low temperatures with warming occurring proximate to the time of administration to preserve efficacy and potency. For example, in mRNA-based SARS-CoV-2 vaccines, the material must be stored at temperatures between minus 20 degrees Celsius and minus 70 degrees Celsius depending on vaccine composition. Such storage temperature requirements can pose challenges for the formulation, implementation and use of RNA based materials in therapeutic settings particularly if the therapeutic is administered on a routine basis, for example daily.

There is a continuing need for better and more robust RNA storage and delivery methods as well as compositions that can facilitate the same. To this end, the concept of using Ca, Mg or mixtures thereof have been contemplated as a bioabsorbable substrate. Both Ca and Mg are present in the body in high concentrations and break down easily. For example, it has been contemplated to use Mg alloys as orthopedic screws.

The concept of using magnetic beads to isolate nucleic acids from biological samples has also been contemplated in analytical settings. Beads composed of material such as the mineral oxide FeO has been employed to isolate biological molecules such as single-strand nucleic acid molecules from mixtures. Additionally, nanoparticles composed of materials such as SiO coated over a magnetic FeO core have been employed to isolate nucleic acids for research purposes. In hospital settings nucleic acid isolation SiO coated magnetic FeO nanoparticles have been employed to isolate nucleic acids for disease diagnosis, prognosis and in safety testing.

Thus, it would be desirable to provide a composition that could be employed to store and transport single strand nucleic acid material that can deliver significant quantities of the stored material, particularly storage in a manner that does not require extreme environmental storage conditions. It would also be desirable to provide a composition and method for storage of single strand nucleic acid material that has increased biocompatibility with biological material and life forms.

### SUMMARY

Disclosed is a composition comprising: a) a plurality of biocompatible nanospheres, the biocompatible nanospheres each having an outer surface with elemental carbon connected thereto; b) a plurality of single strand nucleic acid, wherein at least a portion of the individual single strand nucleic acids are in coordinated connection with the biocompatible nanospheres; and c) a carrier medium.

Also disclosed is a method for storing and/or transporting and/or delivering single strand nucleic acid that includes the steps of forming biocompatible aggregates of nucleic acid and biocompatible nanospheres in which the biocompatible nanospheres employed have an outer surface with elemental carbon connected thereto. In the biocompatible aggregates from that are formed, the single strand nucleic acid is complexed with one or more nanospheres at one or more locations on the single strand nucleic acid in a manner that minimizes the degrees of freedom available for the single strand nucleic acid. In certain embodiments, the single strand nucleic acid present in the biocompatible aggregate formed will be connected to at least two biocompatible nanospheres.

### BRIEF DESCRIPTION OF THE DRAWINGS

FIG. 1 depicts a representation of the interaction between the elemental carbon layer of an embedment of the biocompatible nanosphere as disclosed herein and a heteroatom portion of an associated single strand nucleic acid.

FIG. 2 depicts theoretical bonding energy versus distance for the configuration of FIG. 1.

FIG. 3 depicts a theoretical representation of a representative embodiment of a biocompatible aggregate as disclosed herein.

FIG. 4 is a photomicrograph of an embodiment of the biocompatible aggregate as disclosed herein.

FIG. 5 is a side-by-side photograph showing a sample with unbound RNA and unbound RNA exposed to ribonuclease.

FIG. 6 is a photographic depiction of bound and unbound RNA samples.

FIG. 7 is directed to stability data for RNA present in urine samples.

FIG. 8 is a graphic and tabular depiction of RNA stability in shipping and storage.

### DETAILED DESCRIPTION

While the disclosure has been described in connection with certain embodiments, it is to be understood that the disclosure is not to be limited to the disclosed embodiments and is intended to cover various modifications and equivalent arrangements included within the scope of the appended claims, which scope is to be accorded the broadest interpretation to encompass all such modifications and equivalent structures as is permitted under the law.

Disclosed herein is a composition suitable for the storage and/or transport and/or delivery of single strand nucleic acid in an efficient and economic manner. In certain situations, the single strand nucleic acid can be a ribonucleic acid such as biologically active RNA involved in protein synthesis. Non-limiting examples of such single strand nucleic acid include messenger RNA (mRNA), transfer RNA (tRNA), ribosomal RNA (rRNA), small nuclear RNA (smRNA) and the like as well as fragments thereof. It is also contemplated that the composition as disclosed can be employed to facilitate one or more of storage, transport and/or delivery of various other types of RNA including, but not limited to, RNAs involved in processes such as post-transcriptional modification or DNA replication as well as various regulatory RNAs, parasitic RNAs and the like.

The composition as disclosed can store and maintain desired the single strand nucleic acid in a stable manner the permits delivery of elevated quantities of active intact single strand nucleic acid for subsequent use. As used herein, the term “active intact single strand nucleic acid” means nucleic acid capable of performing the intended or desired coding function when delivered for an end-use application. Also disclosed is a method for storing and transporting single strand nucleic acid material in a manner that maintains and delivers elevated quantities of active intact single strand nucleic acid for subsequent use.

The present disclosure is predicated, at least in part, on the unexpected discovery that single strand nucleic acid could be connected to nanoparticulate structures composed of suitable biocompatible substrate in a manner that provides for secure storage and transport of the associated nucleic acid strand material for prolonged periods and/or at moderate temperature. It is also contemplated that in certain embodiments, the single strand nucleic acid, when aggregated with the nanoparticulate structure, can be stably dispersed in the associated carrier fluid with few, if any, dispersion aids. It is also contemplated that the single strand nucleic acid, when aggregated with the nanoparticulate structure, will result in a composition that will be shelf stable at a temperature between 0° C. and 25° C. in certain embodiments.

The nanoparticulate structures present in the composition can be composed of a substrate material having an outer surface with elemental carbon overlying and connected thereto. Wherein desired or required, the outer surface of the nanoparticulate structures can have include at least one rounded region. In certain embodiments, the substrate material can be a configured in a suitable geometry such as particles or beads. In certain embodiments, the substrate will be configured as beads or spheroid bodies. Nanospherical substrates can be employed in certain embodiments.

The substrate material can be composed in whole or in part of biocompatible substrate materials, silica, polymeric materials and the like. In certain embodiments, the substrate material can be a biocompatible metal material. Suitable biocompatible substrate materials can be those which can be introduced into contact with biological life forms such as humans and the like without adverse effects to the life form. In certain embodiments, the biocompatible substrate material can be biologically compatible transition metals, biologically acceptable alkali earth metals and the like. In certain embodiments, the substrate material can be Fe, Co, Ni, Mg, Zn, or Ca as well as mixtures of any of the foregoing. In certain embodiments, the biocompatible metal material can be selected from the group consisting of Fe, Co, Ni, Mg, Zn, Ca and mixtures thereof.

The substrate material can be nanoparticulate in size. In certain embodiments, the biologically compatible nanospheres can have a diameter between 1 and 100,000 nm and between 20 nm and 100,000 nm in certain embodiments. It is also contemplated that the nanospheres can have an average particle diameter between 20 and 100,000 nm and between 50 and 200 nm in certain embodiments.

In the composition as disclosed, at least a portion of the outer surface of the substrate material has elemental carbon in overlying relationship and connecter thereto. The elemental carbon can be composed of or derived from any suitable source, including but not limited to pyrolytic carbon, graphite, graphene as well as mixtures thereof. In certain embodiments, the elemental carbon can surround the substrate in a generally seamless conformal coating.

The elemental carbon coating connected to the substrate may have a thickness suitable to facilitate the interaction between the substrate structure and the single strand nucleic acid. In certain embodiments, the thickness of the carbon coating layer can be between 1 and 40 atomic layers. In some implementations, the elemental carbon connected to the biocompatible nanospheres is present at a thickness between 1 Angstrom and 0.5 nm. Where desired or required, the elemental carbon present in the carbon layer can be derived in whole or in part from one or more graphene, graphite, or pyrolytic carbon. As the terms are employed herein, graphene is taken to be an allotrope of carbon consisting of a two-dimensional hexagonal lattice and graphite consists of stacked layers of graphene. Pyrolytic carbon is similar to graphite but with some covalent bonding between its graphene sheets. More specifically, the elemental carbon may be formed in sheets similar to graphene or graphitic carbon. The elemental coating material may also include certain amounts of graphitic oxide. As used herein, graphitic oxide is defined one or more of the foregoing elemental carbon materials exhibiting carboxylic acid groups and hydroxide groups formed at imperfections in the graphene sheet surface.

Configuration of biocompatible substrate material and elemental carbon can form biocompatible nanospheres. The amount of biocompatible substrate material present in a given biocompatible nanosphere will be the amount necessary to support the carbon layer and provide suitable geometric contour to the biocompatible substrate material such that the nanoparticles possess suitable spherical or semispherical contour. The amount of biocompatible substrate material can be present in the biologically compatible nanospheres will be that sufficient to support the elemental carbon adherent the outer surface while maintaining the resulting nucleic acid aggregates in suitable suspension in an associated carrier medium. In the composition as disclosed. In the composition as disclosed, the biocompatible substrate can compose at least 10% by weight of the biocompatible nanosphere Where desired or required, the biocompatible nanospheres having elemental carbon attached thereto can be composed of between 10% and 99% by weight biocompatible substrate material with the balance being elemental carbon. In certain embodiments, the biocompatible substrate material can be present in an amount between 20% and 95%; between 35% and 95%; between 50% and 95%; between 75% and 95%.

Carbon-coated substrate material can be prepared by any suitable method that will impart a layer of elemental carbon onto at least a portion of the outer surface of the associated substrate. The imparted elemental carbon imparted on the outer surface of the substrate can have a thickness of a little as one atom in certain configurations. It one non-limiting example of a method for preparing such material is outlined in WO2015095398A1 which is disclosed herein by reference. It is contemplated that carbon coated materials can be prepared by positioning a laser is incident on target in a solvent such that the pulsed laser produces laser pulses having a pulse duration greater than 1 ps at a wavelength between 200 nm and 1500 nm at a pulse repetition rate of at least 10 Hz and a fluence greater than 10 J/cm². The laser beam may be scanned across the surface of the target, i.e., the desired core material (Silica, magnetic metal and the like). The liquid in which the target submerged is typically transparent to laser irradiation of the wavelength being used and is typically a carbon containing solvent such as xylenes or toluene. The carbon shell is fabricated as a result of the laser process. This process results in the seamless and conformal coating of these particles with a carbon layer which has affinity only for single stranded nucleic acids.

The elemental carbon overlying and/or connected to the outer surface of the biocompatible substrate material can be spherical. The biocompatible nanospheres employed in the composition as disclosed herein can have elemental carbon attached to the outer surface of the substrate material in a suitably conformal manner with at least portion of the benzene dimers present in the elemental carbon layer oriented in a manner that flat lattice orientation relative to the substrate surface.

The resulting biocompatible nanospheres in the composition as disclosed can have an average diameter sufficient to maintain the spheres in suspension in the resulting composition as desired or required. In certain embodiments, the biocompatible nanospheres have an average diameter between 1 nm and 500 nm. In certain embodiments, the biocompatible nanospheres have an average diameter between 1 nm and 250 nm.

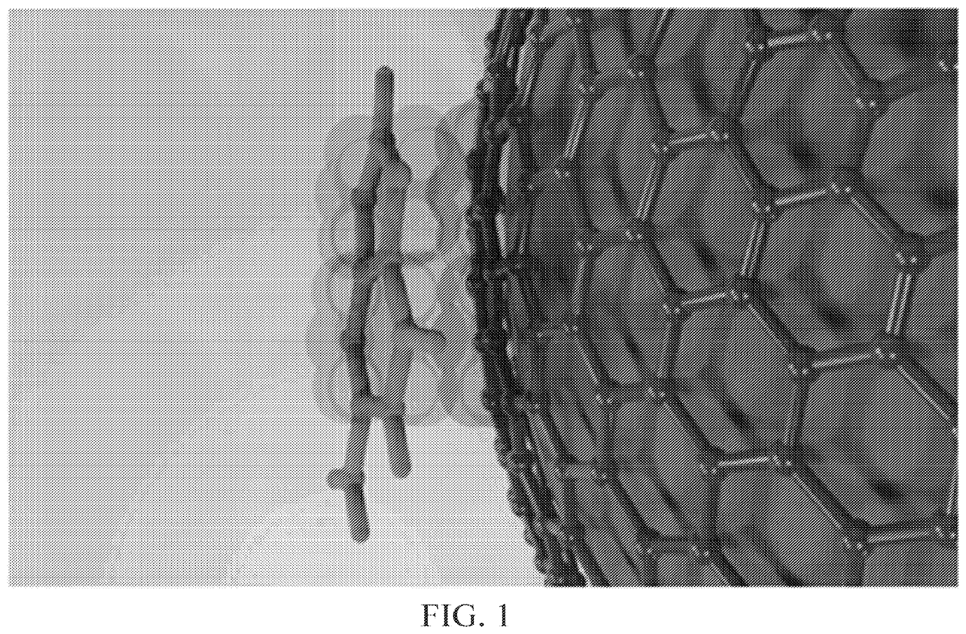

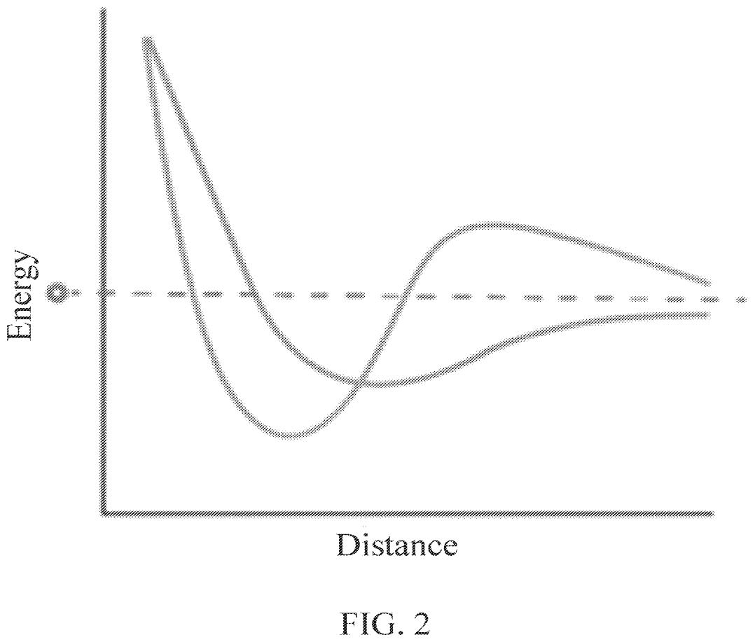

Without being bound to any theory, it is believed that this orientation facilitates pi-pi stacking with one or more aromatic groups aromatic groups present in the single-strand nucleic acid compounds also present in the composition, particularly those present int one or more base pairs present in the single strand nucleic acid or fragment thereof. The pi-pi stacking facilitated can be sandwich, T-shaped or parallel displaced. A representation of the interaction between the elemental carbon layer and a heteroatom portion of an associated single strand nucleic acid is illustrated in FIG. 1. Theoretical bonding energy versus distance is depicted in FIG. 2.

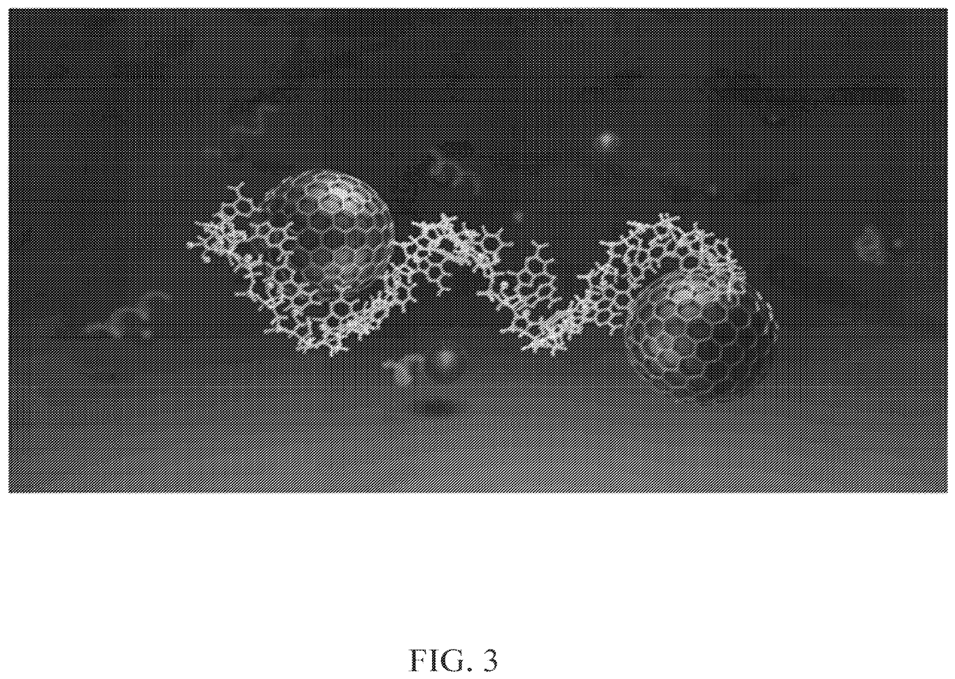

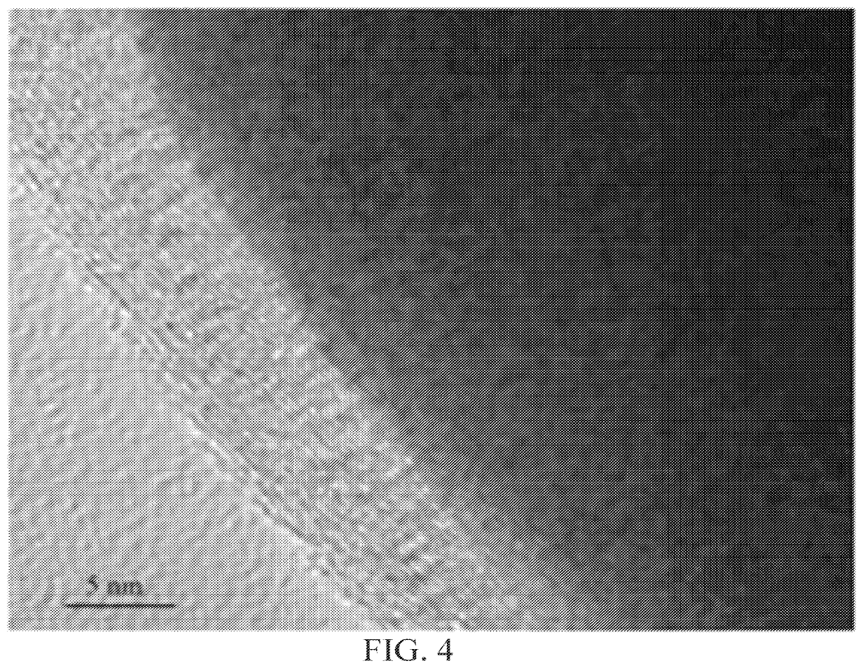

In the composition as disclosed herein, individual single nucleic acids strands and individual biocompatible nanospheres interact to form nanoparticle aggregates with at least a portion of the single strand nucleic acid. In certain embodiments, aggregates include an individual single nucleic strand acids in coordinated connection with one or more individual biocompatible nanospheres. In certain embodiments, the individual nucleic acid strands in coordinated connection with at least two individual biocompatible nanospheres are oriented such that a segment of the respective individual single nucleic acid strands are each located between the at least two respective biocompatible nanospheres are in an unconnected state. A theoretical representation of the biocompatible aggregate is illustrated in FIG. 3, while a photomicrograph of the material is depicted in FIG. 4.

When the biocompatible nanospheres as disclosed interact to form biocompatible nanoparticle aggregates. The biocompatible nanoparticle aggregates can have a size between sufficient to remain dispersed in an associated carrier medium. In certain embodiments, the biocompatible nanoparticle aggregates can have a size between 50 and 750 nm, with sizes between 50 nm and 500 nm in certain embodiments.

The carrier medium employed in the composition as disclosed herein will be one that is compatible with nucleic acid compounds and can facilitate long term storage of the aggregates of the single strand nucleic acid and nanospheres. The carrier medium can be a fluid or liquid (e.g., an aqueous) carrier medium that permits the general or partial dispersion and/or suspension of the aggregates of the single strand nucleic acid and nanospheres therein.

The carrier medium chosen can be a suitable fluid material such as a biocompatible liquid. Suitable biocompatible liquids can include water and/or biocompatible organic fluids as well as mixtures thereof. In certain embodiments, the carrier medium can include suitable biocompatible suspension aids and the like.

The carrier medium can be one that is capable maintaining the single strand nucleic acid present in the biocompatible nanoparticles in essentially intact state that is stabilized against RNase activity. The carrier medium can be composed of suitable aqueous fluids, organic fluids, and mixtures thereof. Where desired or required, the carrier can be composed of water optionally with one or more natural or synthetic lipids, buffers, and electrolytes present. It is within the purview of this disclosure that the additive materials can be altered depending on specific storage and transport needs.

Non limiting examples of suitable lipids include compounds such as ((4-hydroxybutyl)azanediyl)bis(hexane-6,1-diyl)bis(2-hexyldecanoate), polyethylene glycol[PEG] 2000 dimyristoyl glycerol, 1,2-distearoyl-sn-glycero-3-phosphocholine and the like.

The carrier medium can include biologically acceptable Group II ions. These ions can be obtained from biologically suitable Group II ionic materials. In certain embodiments, the biologically suitable Group II ionic materials are present in an amount sufficient to enhance binding between the single-stranded nucleic acid and biocompatible nanospheres. In certain embodiments, the biologically suitable Group II ionic materials can be present at a concentration of 10 mM to 1000 mM; 50 mM to 1000 mM; 100 mM to 1000 mM; 200 mM to 1000 mM; 500 mM to 1000 mM; 700 mM to 1000 mM; 10 mM to 500 mM; 50 mM to 500 mM; 100 mM to 500 mM; 200mM to 500 mM; 250 mM to 500 mM; 300 mM to 500 mM; 400 mM to 500 mM; 10 mM to 400 mM; 50 mM to 400 mM; 100 mM to 400 mM; 150 mM to 400 mM; 200 mM to 400 mM; 250 mM to 400 mM; 300 mM to 400 mM; 350 mM to 400 mM; 10 MM to 250 mM; 50 mM to 250 mM; 100 mM to 250 mM; 200 mM to 250 mM. Without being bound to any theory, it is believed that at least a portion of the Group II ionic materials can be derived from osmolarity regulating buffers.

Where desired or required, the Group II ionic materials can be selected from the group consisting of Ca²⁺, Mg²⁺, and mixtures thereof. In certain applications such materials can be derived, at least in in part, from suitable lysis buffers. Non-limiting examples of lysis buffers can include calcium ion containing lysis buffers containing calcium ions such as those derived from calcium chloride, magnesium ion lysis buffers containing magnesium ions such as those derived from MgCl₂, as well as mixtures thereof. Without being bound to any theory, it is believed that the biologically suitable Group II ionic material unexpectedly functions to enhance binding between the single strand nucleic acid (e.g., RNA single strands) and the biocompatible nanospheres.

In certain embodiments, the carrier medium may include CaCl₂ at a concentration of 50 mM to 500 mM as a functional component which enhances binding.

The carrier medium may also include ethanol or may be heated as single stranded nucleic acids may form complexes with the carbon more quickly and these components are known to denature nucleic acids to make them single stranded. The carrier medium may also contain chaotropic salts or surfactants as necessary to liberate nucleic acids for binding to the beads. For example, in many applications like diagnostic urine testing, the nucleic acids may be inside of cells and liberation of the material from these cells is required before binding can take place.

Non-limiting examples of such buffers include materials such as guanidinium thiocyanate, ethylenediaminetetraacetic acid (EDTA), tris(hydroxymethyl)aminomethane (Tris), dithiothreitol (DTT), and triton X. In certain embodiments, the binding buffer may be guanidinium thiocyanate, ethylenediaminetetraacetic acid (EDTA), tris(hydroxymethyl)aminomethane (Tris), dithiothreitol (DTT), and triton X and the solution may be adjusted to a pH of 6.5. EDTA and DTT may optionally be present to inactivate proteins and may be present in concentrations from 1 mmol to 100 mmol. Tris may optionally be present as a buffer to maintain the pH at 6.5. The pH may range from 4 to 9. The concentration of tris buffer may be from 20 to 200 mmol. Triton X is a detergent and may optionally be used to homogenize the solution. The concentration of Triton X is from 1 to 50 mmol. In certain embodiments, a suitable binding buffer may only require 1M to 7M guanidinium ions.

When the biocompatible nanospheres as disclosed interact to form biocompatible nanoparticle aggregates, the resulting biocompatible nanoparticle aggregates can have a size sufficient to remain dispersed in an associated carrier medium. In certain embodiments, the biocompatible nanoparticle aggregates can have a size between 50 and 1000 nm, with sizes between 50 nm and 500 nm; between 50 nm and 400 nm; 50 nm and 300nm; in certain embodiments.

Without being bound to any theory, it is believed that the biocompatible aggregates composed of the single strand nucleic acid and the biocompatible nanosphere exhibit some specific bonding patterns when present in the composition as disclosed herein. The interaction between the single strand nucleic acid and the biocompatible nanoparticles as disclosed herein constrains the ability of the associated nucleic acid strand to react with other regions on the nucleic acid strand or other reactive compounds that may be present or introduced into the composition.

Without being bound to any theory, it is believed that at least a portion of the bonding in or attraction between the biocompatible nanospheres and the single strand nucleic acid occurs between the lattice structure of the elemental carbon layer benzene and/or pyridine moieties in the present in the nucleic acid structure evidenced in the pi-pi bonding.

It is also believed that the size of the biocompatible nanospheres relative the length of many types of single strand nucleic acid is such that two or more biocompatible nanospheres can be bound to or associated with a given nucleic acid strand, further constraining unhindered movement of the associated nucleic acid strand.

The resulting composition of carrier medium containing aggregates of single strand nucleic acid and one or more biocompatible nanospheres with an outer surface having elemental carbon in overlying relationship thereon provides a stable storage medium for the associated single strand nucleic acid material.

It has been found, quite unexpectedly, that the single strand nucleic acid such as messenger RNA (mRNA) when present in the composition as disclosed is shelf stable as a suspension at temperatures between −20° C. and 25° C.; when required for administration or use, the biocompatible aggregate and be employed directly or the single strand nucleic acid material can be separated from contact with the biocompatible nanosphere by any suitable means.

In addition to temperature stability, it is believed that the composition as disclosed herein exhibits shock resistance as well as providing a means for storage and transport of single strand nucleic acid material in a manner that preserves the activity of the associated RNA and minimizes chain breakage and impairment.

Also disclosed is a method for storing and/or transporting single strand nucleic acid material. The method as disclosed can be used to maintain single strand nucleic acid material in a stable or essentially stable form for storage as well as transport between locations. The single strand nucleic acid material suitable for sub storage methods include but is not limited to can be ribonucleic acid (RNA). In certain embodiments, the RNA to be stored can be messenger RNA (mRNA), transfer RNA (tRNA), ribosomal RNA (rRNA), small nuclear RNA (smRNA), and the like as well as fragments thereof.

In the method disclosed, biocompatible aggregates of single strand nucleic acid material and biocompatible particles such as nanospheres are formed such that individual strands of the single strand nucleic acid material are connected to at least one biocompatible nanosphere in a manner that constrains the freedom of movement of the individual nucleic acid strands. It is contemplated that at least a portion of the single strand nucleic acid material will be bound with two or more nanospheres. The biocompatible aggregates that are formed can be maintained in a suitable suspension or dispersion composed of one or more carriers.

The biocompatible nanoparticles such as nanospheres employed will each be composed a suitable biocompatible material and have an outer surface with elemental carbon connected thereto. The elemental carbon surface layer can be composed of a material of sufficient thickness and structure to induce and support pi-pi bonding between aromatic ring structures present in the nucleic acid strand material and the carbon lattice structure present in the elemental carbon layer. In certain embodiments, the elemental carbon material in the layer will be composed in whole or in part of one or more compounds such as graphene, graphite, pyrolytic carbon, and the like. In certain embodiments, the elemental carbon layer will be graphene.

The biocompatible substrate material can be a suitable biologically compatible material to which the elemental carbon can bond or be otherwise connected. In certain embodiments, the biocompatible substrate material can be biologically compatible transition metals, biologically acceptable alkali earth metals and the like. In certain embodiments, the substrate material can be Fe, Co, Ni, Mg, Zn as well as mixtures of any of the foregoing. In certain embodiments, the biocompatible metal material can be selected from the group consisting of Fe, Co, Ni, Mg, Zn, and mixtures thereof.

The element carbon layer can be connected to the biocompatible substrate material in any suitable manner. Non-limiting examples of such connection methods include boning, mechanical deposition, and the like. The elemental carbon layer can have a thickness suitable to promote interaction between the lattice structure present in the carbon layer and specific aromatic functionalities present in the nucleic acid strand material. In certain embodiments, the elemental carbon will be present in layered thickness between 1 and 40 atomic layers.

It is contemplated that the biocompatible substrate material can be maintained in a suitable carrier medium that is typically a fluid carrier medium such as water organic fluids or mixtures thereof. The carrier medium can be one the facilities dispersion of the biocompatible nanoparticles in a manner that facilitates uptake of the single strand nucleic acid material and stable storage of the same.

The biocompatible aggregates formed during the forming step can be produced by a process of introducing the single strand nucleic acid material into contact with the biocompatible nanospheres. The single strand nucleic acid material can be produced and or purified by any suitable means or method. It is contemplated the single strand nucleic acid material that is introduced can be homogenous or essentially homogeneous if desired or required.

Introduction of single strand nucleic acid into the carrier can occur by any suitable method and proceed in a manner that achieves aggregation. In certain embodiments it is contemplated that the formation step can occur under certain solution conditions that facilitate such uptake.

The method can also include the step of maintaining the biocompatible aggregates of single strand nucleic acid and biocompatible nanospheres at a temperature that maintains the viability and activity of the single strand nucleic acid present in the biocompatible aggregate. In certain embodiments, the temperature can be between −20° C. and 30° C. during the storage interval, with temperatures between 0° C. and 20° C. being employed in certain embodiments.

The biocompatible aggregates thus formed can be transported from location to location in the aggregated state and can be subjected to additional operations post storage as desired or require.

Where desired or required, the method can also include the step of delivering the biocompatible aggregates to a biological destination after expiration of the storage interval. In certain embodiments, the biocompatible aggregates may be delivered directly to the intended destination. In other embodiments, the single strand nucleic acid may be separated from the nanospheres and then delivered to the intended site or destination.

The following examples are provided to illustrate the invention but are not intended to limit the scope thereof. All parts and percentages are by weight unless otherwise indicated.

### EXAMPLE 1

Biocompatible nanospheres composed of one of various biocompatible metals having an average diameter between 5 nm and 250 nm and an elemental carbon layer of a thickness of 1 to 40 carbon atoms are admixed in an aqueous solution. Five different biocompatible substrate materials are evaluated: Fe, Co, Ni, Mg, Ca, Zn, and mixtures thereof. The most exceptional examples are Fe65Co35 and Ca. Core materials of Fe65Co35 are utilized for applications involving diagnostics. This is because this allows the material to be separated from the solution matrix with a magnet. The other exceptional example is Ca, which allows the material to be absorbed by the human body and is most useful for therapeutic applications.

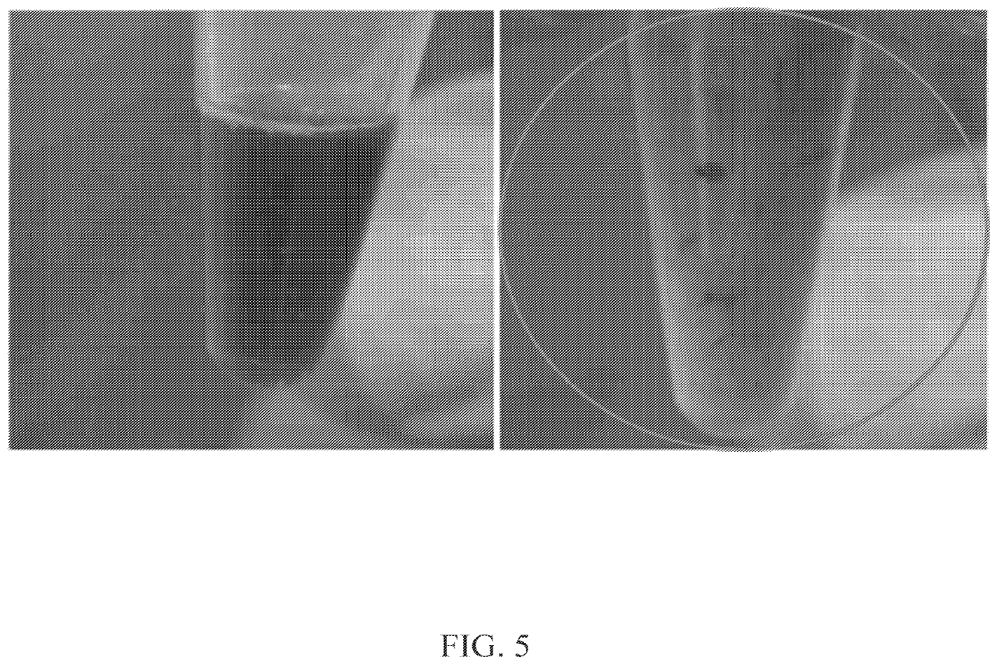

The respective biocompatible nanospheres are each admixed in an 18 ml of aqueous carrier fluid each in an amount of 20 mg of nanospheres at a temperature of 25° C. To an aliquot of single strand nucleic acids, 70 ul of the nanosphere solution is introduced. Binding is ascertained to have occurred by visual inspection, where binding is visible by formation of clusters of particles as in FIG. 5, or quantification by an instrument such as a Denovix Nanodrop, where a fraction of the aliquot of nucleic acids can be analyzed for the presence of nucleic acids via light absorption at the wavelength of 260 nm. In the original nucleic acids solution, significant absorption of light at 260 nm indicates the presence of nucleic acids. After binding, this signal disappears as the nucleic acids are no longer present in solution, indicating biocompatible aggregate formation.

### EXAMPLE 2

The stability of biocompatible aggregates formed by the process outlined in Examples 2 present in a carrier medium composed of RNase free water at a pH of 10 was examined by exposing a 2 ml portions of carrier medium having biocompatible aggregates. A control having a concentration of single strand nucleic acid at 5 ug/ml was also prepared. The sample materials are maintained at a temperature of 25° C.

The samples were shaken to ensure thorough mixing, the samples were analyzed to determine RNA integrity at 1 day, 5 days and 25 days by capillary gel electrophoresis to determine the relevant RNA integrity number (RIN).

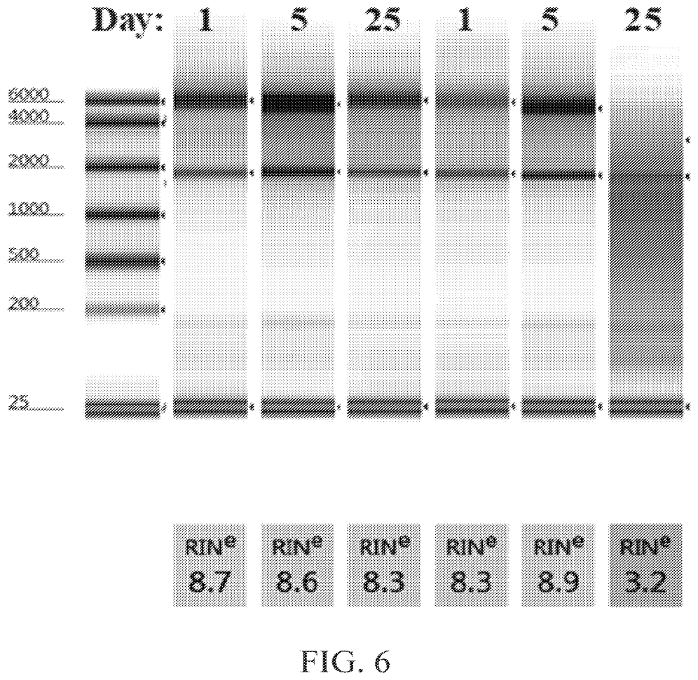

FIG. 5 is a side-by-side photograph showing a sample with unbound RNA in RNase free water and RNA bound in biocompatible aggregate. The visible clumping evident in the RNA-bound material is evidence of the efficiency of RNA binding efficiency. The RIN and associated gel electrophoresis charts are set forth in FIG. 6.

The Day 1 and Day 5 samples of biocompatible aggregate material and the Day 1 and Day 5 unbound material each have RINs above 8. Variation occurs in the Day 25 data in which the biocompatible aggregate material has a RIN above 8 while the free RNA not in a biocompatible aggregate has a RIN value of 3.2, indicating the long-term storage capacity on the composition and method as disclosed herein. One example of how this is industrially relevant is in the distribution of mRNA vaccines. These normally need a cold chain of custody to maintain RNA integrity as this demonstrates as even in pure water the RNA will degrade after 25 days. The biocompatible aggregates maintain integrity at room temperature for at least 25 days.

### EXAMPLE 3

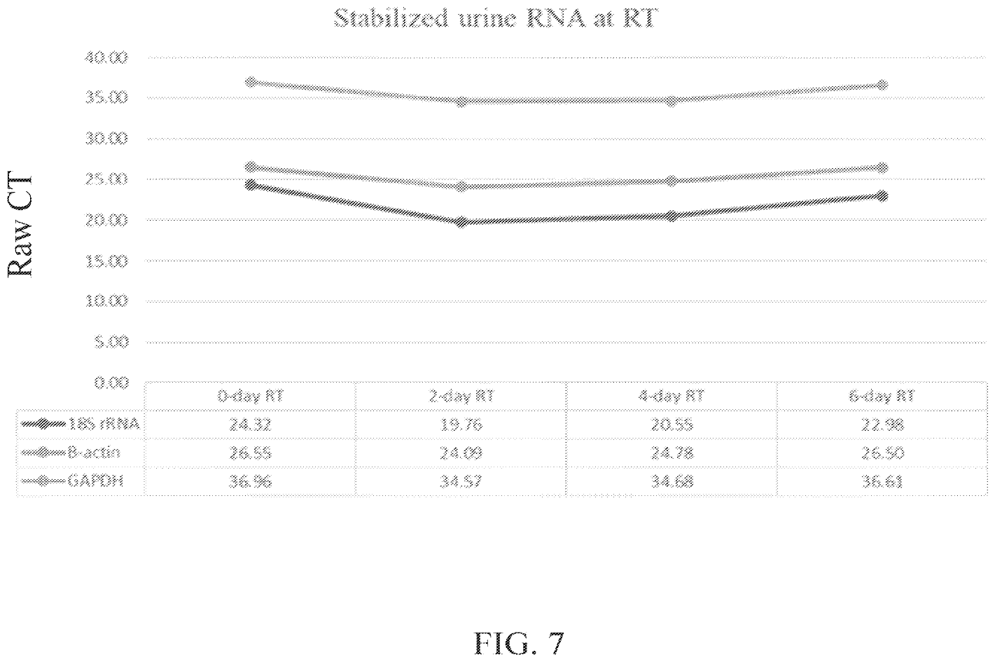

To further analyze the stability of the composition and method as disclosed therein, urine RNA is stabilized in the manner describe herein at room at room temperature monitoring glyceraldehyde-3-phosphate dehydrogenase (GADPH) activity 702, β-actin activity 704 as well as 18s rRNA 706 a6s Day 0, Day 2, Day 4, and Day 6. The data is depicted graphically and tabularly in FIG. 7. Stability of RNA collected from urine is important for applications such as at-home kidney transplant rejection testing and prostate cancer screening.

### EXAMPLE 4

The composition as disclosed Example 1 was employed to collect and transport RNA derived from urine.

To evaluate the storage and transport capacity of the composition as disclosed herein as urine sample is introduced into a sample vial with suitable enzymes to achieve lysis to which biocompatible nanospheres having an elemental carbon coating on an Fe substrate are added. The vial is then placed in a magnetic holder and the liquid is removed. Ethanol is then added to the vial and then seal and shipped. The material was maintained at room temperature.

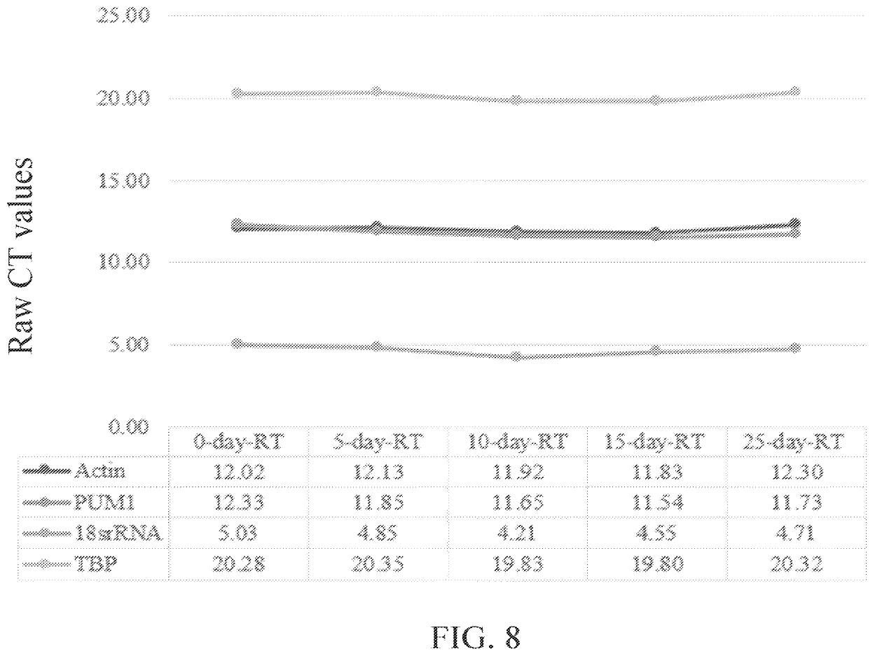

Activity of the stored RNA was tested at initiation, at Day 5, Day 10, Day 15, and Day 25 for activity relative to Pumillio homolog 1 (PUM1) 802, Actin 804, 18S ribosomal RNA (18srRNA) 806, and TATA-bonding protein (TBP) 808. The activity values did not vary throughout the testing interval, demonstrating that the single strand RNA remained associated with the biocompatible aggregate. The results are summarized in FIG. 8.

While the invention has been described in connection with certain embodiments, it is to be understood that the invention is not to be limited to the disclosed embodiments but, on the contrary, is intended to cover various modifications and equivalent arrangements included within the scope of the appended claims, which scope is to be accorded the broadest interpretation to encompass all such modifications and equivalent structures as is permitted under the law.

1. A composition, comprising:
a plurality of biocompatible nanospheres, the biocompatible nanospheres each having a biocompatible substrate having an outer surface with elemental carbon connected thereto;
a plurality of individual single strand nucleic acids, wherein at least a portion of the individual single strand nucleic acids are in coordinated connection with the biocompatible nanospheres; and
an aqueous carrier medium, wherein:
individual single nucleic acids strands of the plurality of single strand nucleic acids and individual biocompatible nanospheres interact to form biocompatible nanoparticle aggregates,
at least a portion of the biocompatible nanoparticle aggregates include individual single nucleic strand acids in coordinated connection with at least two individual biocompatible nanospheres,
the biocompatible substrate is present in the biocompatible nanospheres in an amount of at least 10% by weight, and
a coordinated connection between the single strand nucleic acid and the elemental carbon exhibits pi-pi bonding.

2. (canceled)

3. The composition of claim 1, wherein the individual single nucleic acid strands in coordinated connection with at least two respective biocompatible nanospheres are oriented such that a segment of the respective individual single nucleic acid strands are each located between the at least two respective biocompatible nanospheres and are in an unconnected state.

4. The composition of claim 1, wherein at least one of the individual single strand nucleic acids is RNA.

5. The composition of claim 1, wherein the biocompatible nanospheres comprise a biocompatible substrate material, the biocompatible substrate material selected from the group consisting of Fe, Co, Ni, Mg, Zn, and mixtures thereof.

6. The composition of claim 1, wherein the biocompatible nanospheres comprise carbon.

7. The composition of claim 1, wherein the elemental carbon connected to the biocompatible nanospheres is present at a thickness between 1 angstrom and 0.5 nm.

8. The composition of claim 1, wherein the biocompatible nanospheres have an average diameter between 1 nm and 500 nm.

9. The composition of claim 8, wherein the biocompatible nanospheres have an average diameter between 5 nm and 250 nm.

10. The composition of claim 1, wherein the elemental carbon is present as a conformal coating.

11. The composition of claim 10, wherein the elemental carbon has a thickness of one to 40 atomic layers.

12. The composition of claim 10, wherein the elemental carbon is formed in sheets to of graphene or graphitic carbon.

13. The composition of claim-2 1, wherein the nanoparticle aggregates have a size between 50 and 750 nm.

14. (canceled)

15. (canceled)

16. The composition of claim 1, further comprising biologically acceptable Group II ions selected from the group consisting of calcium ions, magnesium ions and mixtures thereof and present in an amount between 50 mM to 500 mM.

17. (canceled)

18. (canceled)

19. (canceled)

20. A composition for storage of RNA, the composition comprising:
a plurality of biocompatible nanospheres, the biocompatible nanospheres each having a biocompatible substrate having an outer surface with elemental carbon connected thereto, the biocompatible nanospheres having an average diameter between 1 nm and 500 nm and wherein the elemental carbon is present as a conformal coating, wherein the biocompatible substrate is present in the biocompatible nanospheres in an amount of at least 10% by weight;
an aqueous carrier medium; and
biologically acceptable Group II ions present at a concentration sufficient to enhance binding between RNA single strands and the biocompatible nanospheres and present in an amount between 50 mM to 500 mM.

21. The composition of claim 20, wherein the biologically acceptable Group II ions are selected from the consisting of calcium ions, magnesium ions, and mixtures thereof.

22. (canceled)

23. (canceled)

24. The composition of claim 20, wherein the biocompatible nanospheres have an average diameter between 5 nm and 250 nm.

25. The composition according to claim 20, wherein the elemental carbon has a thickness of one to 40 atomic layers and is formed in sheets of graphene or graphitic carbon.

26. The composition of claim 25, wherein the elemental carbon connected to the biocompatible nanospheres is present at a thickness between 1 Angstrom and 0.5 nm.

27. A composition, comprising:
a plurality of biocompatible nanospheres, the biocompatible nanospheres each having an outer surface with elemental carbon connected thereto, and the biocompatible nanospheres having an average diameter between 1 nm and 500 nm, and wherein the elemental carbon is present as a conformal coating;
a plurality of individual single strand nucleic acids, wherein at least a portion of the individual single strand nucleic acids are in coordinated connection with the biocompatible nanospheres, wherein the plurality of individual single strand nucleic acids is-are RNA;
a liquid an aqueous carrier medium; and
biologically acceptable Group II ions selected from the group consisting of calcium ions, magnesium ions and mixtures thereof and present at a concentration sufficient to enhance binding between the plurality of individual single strand nucleic acid and the plurality of biocompatible nanospheres,
wherein a portion of the individual nucleic acid strands of the plurality of individual single strand nucleic acids are in coordinated connection with at least two biocompatible nanospheres and are oriented such that a segment of the respective individual single nucleic acid strands are located between the at least two biocompatible nanospheres.

28. (canceled)

29. The composition of claim 27, wherein the biologically acceptable Group II ions are present in an amount between 50 mM to 500 mM.

30. (canceled)

31. The composition of claim 27, wherein the biocompatible nanospheres have an average diameter between 5 nm and 250 nm.

32. The composition of claim 27, wherein the elemental carbon has a thickness of one to 40 atomic layers and is formed in sheets of graphene or graphitic carbon present at a thickness between 1 Angstrom and 0.5 nm.

33. (canceled)

34. The composition of claim 32, wherein the biocompatible nanospheres are composed of biocompatible metal material, the biocompatible metal material selected from the group consisting of Fe, Co, Ni, Mg, Zn, and mixtures thereof.

35. The composition of claim 34, wherein the coordinated connection between the single strand nucleic acid and the elemental carbon includes pi-pi bonding.

36. The composition of claim 35, wherein individual single nucleic acids strands and individual biocompatible nanospheres interact to form nanoparticle aggregates, wherein at least a portion of the biocompatible aggregates include an individual single nucleic strand acid in coordinated connection with at least two individual biocompatible nanospheres.

37. A method for storing and transporting single strand nucleic acid material, comprising the steps of:
forming biocompatible aggregates of single strand nucleic acid and biocompatible nanospheres in an aqueous carrier medium, wherein the biocompatible aggregates include biocompatible nanospheres each having an outer surface with elemental carbon connected thereto and individual strands of nucleic acid connected to at least two of the biocompatible nanospheres.

38. The method of claim 37, further comprising the step of maintaining the biocompatible aggregates of single strand nucleic acid and biocompatible nanospheres at a temperature between 0 degrees and 30 degrees Celsius during a storage interval.

39. The method of claim 38, further comprising the step of delivering the biocompatible aggregates to a biological destination after expiration of the storage interval.

40. The method of claim 38, further comprising the step of separating the single strand nucleic acid from contact with the biocompatible nanospheres after expiration of the storage interval.

41. The method of claim 37, wherein the biocompatible nanospheres have an average diameter between 1 nm and 500 nm, and wherein the elemental carbon is present as a conformal coating.

42. The method of claim 37, wherein the elemental carbon has a thickness of one to 40 atomic layers and is formed in sheets similar to graphene or graphitic carbon.

43. The method of claim 42, wherein the elemental carbon connected to the biocompatible nanospheres is present at a thickness between 1 Angstrom and 0.5 nm.

44. The method of claim 37, wherein forming the biocompatible aggregates proceeds in a composition that includes the biocompatible nanospheres, and the single strand nucleic acids, and further comprises biologically acceptable Group II ions selected from the consisting of calcium ions, magnesium ions, and mixtures thereof.

45. (canceled)
